(19) United States
(12) Patent Application Publication
(10) Pub. No.: US 2025/0104817 A1
(43) Pub. Date: Mar. 27, 2025

(54) TRAINING METHOD AND APPARATUS FOR FULL ATOMIC STRUCTURE PREDICTION MODEL, AND ELECTRONIC DEVICE

(71) Applicant: BEIJING BAIDU NETCOM SCIENCE TECHNOLOGY CO., LTD., Beijing (CN)

(72) Inventors: Lihang Liu, Beijing (CN); Xiaomin Fang, Beijing (CN); Xiaonan Zhang, Beijing (CN); Jingzhou He, Beijing (CN)

(73) Assignee: BEIJING BAIDU NETCOM SCIENCE TECHNOLOGY CO., LTD., Beijing (CN)

(21) Appl. No.: 18/974,497

(22) Filed: Dec. 9, 2024

(30) Foreign Application Priority Data
Sep. 23, 2024 (CN) 2024113311233

(51) Int. Cl. G16C 20/70; G16C 20/30

(52) U.S. Cl. CPC G16C 20/70; G16C 20/30

(57) ABSTRACT

A training method and apparatus for a full atomic structure prediction model. The method includes: obtaining structural information of a biomolecule and a first dynamic trajectory of the biomolecules; in which, the first dynamic trajectory includes position information of atoms in the biomolecule at different time points; adding noise to the first dynamic trajectory to obtain a second dynamic trajectory; encoding the structural information to obtain encoded features; decoding the encoded features and the second dynamic trajectory to obtain a target dynamic trajectory; and training an initial full atomic structure prediction model based on a difference between the target dynamic trajectory and the first dynamic trajectory, to obtain the full atomic structure prediction model.

### CROSS-REFERENCE TO RELATED APPLICATIONS

The present application is based on and claims the priority of Chinese patent application No. 2024113311233 filed on Sep. 23, 2024, the entire contents of which are incorporated herein by reference.

### TECHNICAL FIELD

The disclosure relates to the field of computer technology, in particular to artificial intelligence fields such as deep learning and biological computing. Specifically, it relates to a training method and apparatus for a full atomic structure prediction model, and an electronic device.

### BACKGROUND

With the development of artificial intelligence, the use of artificial intelligence technology for predicting a full atomic structure of a biomolecule has received more attention compared to experimental methods. How to use deep learning methods to promote accurate prediction of the full atomic structure of the biomolecule has become increasingly important.

### SUMMARY

The disclosure provides a training method and apparatus for a full atomic structure prediction model, and an electronic device.

According to an aspect of the present disclosure, a training method for a full atomic structure prediction model is provided, including:
obtaining structural information of a biomolecule and a first dynamic trajectory of the biomolecule; wherein, the first dynamic trajectory includes position information of atoms in the biomolecule at different time points;
adding noise to the first dynamic trajectory to obtain a second dynamic trajectory;
encoding the structural information to obtain encoded features;
decoding the encoded features and the second dynamic trajectory to obtain a target dynamic trajectory; and
training an initial full atomic structure prediction model based on a difference between the target dynamic trajectory and the first dynamic trajectory, to obtain the full atomic structure prediction model.

According to another aspect of the present disclosure, a method for predicting a full atomic structure is provided, including:
obtaining structural information of a biomolecule;
encoding the structural information through a full atomic structure prediction model to obtain encoded features; wherein, the full atomic structure prediction model can be trained using the method described above; and
performing decoding based on the encoded features and dynamic trajectory noise by using the full atomic structure prediction model, to obtain a dynamic trajectory of the biomolecule; wherein, the dynamic trajectory includes position information of atoms in the biomolecule at different time points.

According to another aspect of the present disclosure, an electronic device is provided, including:
at least one processor; and
a memory communicatively coupled to the at least one processor; wherein,
the memory stores instructions executable by the at least one processor, when the instructions are executed by the at least one processor, the at least one processor is enabled to implement the method described in the above embodiments.

According to another aspect of the present disclosure, a non-transitory computer-readable storage medium having computer instructions stored thereon is provided. The computer instructions are configured to enable a computer to implement the method described in the above embodiments.

According to another aspect of the present disclosure, a computer program product is provided, including computer programs, wherein when the computer programs are executed by a processor, steps of the method described in the above embodiments are implemented.

It should be understood that the content described in this section is not intended to identify key or important features of the embodiments of the disclosure, nor is it intended to limit the scope of the disclosure. Additional features of the disclosure will be easily understood based on the following description.

### BRIEF DESCRIPTION OF THE DRAWINGS

The drawings are used to better understand the solution and do not constitute a limitation to the disclosure, in which:

FIG. 1 is a schematic flowchart of a training method for a full atomic structure prediction model provided in an embodiment of the disclosure.

FIG. 2 is a schematic flowchart of a training method for a full atomic structure prediction model provided in another embodiment of the disclosure.

FIG. 3 is a schematic flowchart of a training method for a full atomic structure prediction model provided in another embodiment of the disclosure.

FIG. 4 is a schematic diagram of a process of dynamic trajectory prediction using a full atomic structure prediction model provided in an embodiment of the present disclosure.

FIG. 5 is a schematic flowchart of a method for predicting a full atomic structure provided in an embodiment of the present disclosure.

FIG. 6 is a block diagram of a training apparatus for a full atomic structure prediction model provided in an embodiment of the present disclosure.

FIG. 7 is a block diagram of an apparatus for predicting a full atomic structure provided in an embodiment of the present disclosure.

FIG. 8 is a block diagram of an electronic device used to implement the training method for a full atomic structure prediction model according to embodiments of the disclosure.

### DETAILED DESCRIPTION

The following describes the exemplary embodiments of the disclosure with reference to the accompanying drawings, which includes various details of the embodiments of the disclosure to facilitate understanding, which shall be considered merely exemplary. Therefore, those of ordinary skill in the art should recognize that various changes and modifications can be made to the embodiments described herein without departing from the scope and spirit of the disclosure. For clarity and conciseness, descriptions of well-known functions and structures are omitted in the following description.

The following describes the training method and apparatus for the full atomic structure prediction model, electronic device, and storage medium of embodiments of the present disclosure with reference to the accompanying drawings.

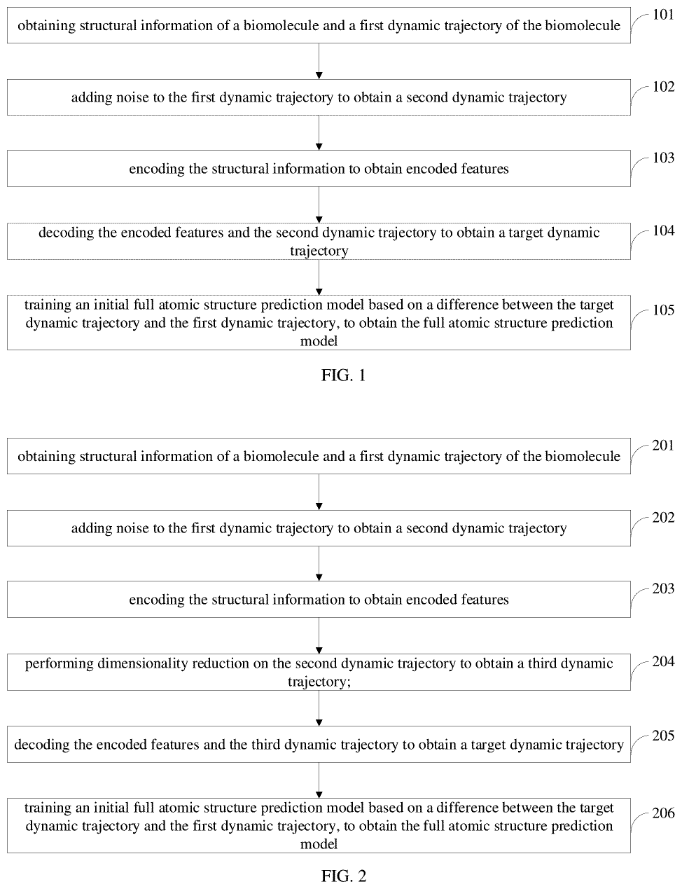

FIG. 1 is a schematic flowchart of a training method for a full atomic structure prediction model provided in an embodiment of the present disclosure.

The training method for the full atomic structure prediction model of embodiments of the present disclosure can be executed by the training apparatus for the full atomic structure prediction model of embodiments of the present disclosure, which can be configured in an electronic device.

The electronic device may be any device with computing power, such as personal computer, mobile terminal, server, etc. The mobile terminal may be a hardware device with various operating systems, a touch screen, and/or a display screen, such as vehicle mounted device, mobile phone, tablet, personal digital assistant, wearable device, etc.

As shown in FIG. 1, the training method for the full atomic structure prediction model includes followings.

In step 101, structural information of a biomolecule and a first dynamic trajectory of the biomolecules are obtained.

In the present disclosure, the biomolecule may be any one of proteins, small molecules, RNA (ribonucleic acid), DNA (deoxyribonucleic acid), ions, etc. The biomolecule may also be a complex of at least two types of proteins, small molecules, RNA, DNA, ions, etc., without limitation.

The structural information of the biomolecule can refer to sequence information, molecular formula, etc. For example, the biomolecule is one of the proteins, RNA, DNA, etc., and structural information can refer to sequence information. For another example, if the biomolecule is a complex of proteins and molecules, structural information can include sequence information of proteins and sequence information of DNA. For yet another example, if the biomolecule is a complex of proteins and small molecules, the structural information can include the sequence information of the protein and the molecular formula of the small molecule.

The first dynamic trajectory may include position information of atoms in the biomolecule at different time points. The position information here can refer to three-dimensional coordinate information. Since the three-dimensional structure of biomolecule is determined by the positions of atoms in the biomolecule, the first dynamic trajectory can be used to characterize the three-dimensional structure of the biomolecule at different time points.

For example, the shape of the first dynamic trajectory can be F1*S1*3, where F1 represents the number of frames, S1 represents the number of atoms in the biomolecule, and 3 represents three dimensions in the three-dimensional coordinate information. It can be considered that one frame corresponds to one time point. The first dynamic trajectory includes the position information of the atoms in the biomolecule at F1 time points, that is, the three-dimensional structure of the biomolecule at F1 time points.

The structural information of the biomolecule corresponds to the first dynamic trajectory. Taking protein sequence information as an example, protein sequence information refers to the arrangement order of amino acids in the protein molecule. The atoms in each frame of the first dynamic trajectory are arranged according to the arrangement order of amino acids, that is to say, the atoms in each amino acid are sorted in sequence according to the arrangement order of amino acids. It should be noted that the arrangement order of atoms remains unchanged at different time points, but the position information of atoms may change.

As an example, the first dynamic trajectory can be based on the three-dimensional structure of the biomolecule obtained at different time points during the experimental phase, or can be obtained using other methods, which is not limited.

In step 102, noise is added to the first dynamic trajectory, to obtain a second dynamic trajectory.

In this disclosure, adding noise to the first dynamic trajectory can obtain a noisy dynamic trajectory, which is the second dynamic trajectory. For example, noise can be added to the position information of atoms in the biomolecule at certain time points from multiple time points, or noise can be added to the position information of some atoms at different time points in the first dynamic trajectory.

The noise used may be Gaussian noise, random noise, or other types of noise.

In step 103, the structural information is encoded to obtain encoded features.

In this disclosure, the initial full atomic structure prediction model may include an encoder and a decoder, and the structural information of the biomolecule can be input into the encoder for encoding and obtaining the encoded features.

In step 104, the encoded features and the second dynamic trajectory are decoded to obtain a target dynamic trajectory.

In this disclosure, the encoded features and the second dynamic trajectory can be input into the decoder of the initial full atomic structure prediction model, and the decoder can be used to decode the encoded features and the second dynamic trajectory to obtain the target dynamic trajectory.

The target dynamic trajectory may include predicted position information of atoms in the biomolecule at different time points.

In step 105, an initial full atomic structure prediction model is trained based on a difference between the target dynamic trajectory and the first dynamic trajectory, to obtain the full atomic structure prediction model.

In this disclosure, the parameters of the initial full atomic structure prediction model can be adjusted based on the difference between the position information of atoms in the biomolecule at each time point in the target dynamic trajectory and the position information of atoms in the biomolecule at the same time point in the first dynamic trajectory, to obtain a full atomic structure prediction model after parameter adjustment. If the training end condition is not met, the full atomic structure prediction model after parameter adjustment can continue to be trained until the training end condition is met, to obtain the full atomic structure prediction model. The full atomic structure prediction model can be used to predict the dynamic trajectory of the biomolecule based on its structural information.

It can be understood that the position information of atoms in the biomolecule at a certain time point can also be used to train and obtain the full atomic structure prediction model that can predict the position information of atoms in the biomolecule at a certain time point, or obtain the full atomic structure prediction model for predicting the static conformation of the biomolecule.

In embodiments of the present application, encoded features are obtained by encoding the structural information of the biomolecule, and the encoded features and the second dynamic trajectory obtained by adding noise to the first dynamic trajectory are decoded to obtain the target dynamic trajectory. Then, based on the difference between the target dynamic trajectory and the first dynamic trajectory, the full atomic structure prediction model is obtained by training. Therefore, based on the structural information of the biomolecule and the first dynamic trajectory of the biomolecule, the full atomic structure prediction model that can predict the dynamic trajectory of the biomolecule is obtained by training, achieving prediction of the dynamic trajectory of the biomolecule and improving the generalization ability of the model.

FIG. 2 is a schematic flowchart of a training method for a full atomic structure prediction model provided in another embodiment of the present disclosure.

As shown in FIG. 2, the training method for the full atomic structure prediction model includes followings.

In step 201, structural information of a biomolecule and a first dynamic trajectory of the biomolecules are obtained.

In this disclosure, step 201 can be implemented using any manner in embodiments of the present disclosure, and will not be elaborated here.

In step 202, noise is added to the first dynamic trajectory, to obtain a second dynamic trajectory.

In this disclosure, step 202 can be implemented using any manner in embodiments of the present disclosure, and will not be elaborated here.

In step 203, the structural information is encoded to obtain encoded features.

In this disclosure, step 203 can be implemented using any manner in embodiments of the present disclosure, and will not be elaborated here.

In step 204, dimensionality reduction is performed on the second dynamic trajectory to obtain a third dynamic trajectory.

In this disclosure, the position information of adjacent atoms in the second dynamic trajectory can be fused to reduce the dimensionality of the second dynamic trajectory and obtain the third dynamic trajectory.

For example, the second dynamic trajectory can be segmented to obtain the third dynamic trajectory. The third dynamic trajectory may include multiple first sub block trajectories. Each first sub block trajectory can be regarded as an element of the third dynamic trajectory, and each first sub block trajectory can be obtained through fusion based on the position information of adjacent atoms in the second dynamic trajectory. The fusion here can be calculating the average, weighted average, or randomly selecting the position information of any atom from the position information of adjacent atoms.

For example, the shape of the dynamic trajectory can be represented as F*S*3, where F represents the number of frames, S represents the number of atoms in the biomolecule, and 3 represents three dimensions in the three-dimensional coordinate information. The number of frames of the first dynamic trajectory is 4 and the number of atoms is 6, which can be regarded as a 4*6 matrix. The matrix can be divided according to 2*2 small blocks to obtain a 2*3 matrix. That is, the number of frames of the second dynamic trajectory is 2 and the number of atoms is 3, with a total of 6 first sub block trajectories. This reduces the dimensionality of the first dynamic trajectory from 4*6*3 to 2*3*3.

In step 205, the encoded features and the third dynamic trajectory are decoded, to obtain a target dynamic trajectory.

In this disclosure, the encoded features and the third dynamic trajectory can be decoded to obtain the intermediate dynamic trajectory, which can then be interpolated to obtain the target dynamic trajectory.

For example, if the decoder of the model processes one-dimensional data, the third dynamic trajectory can be converted into a one-dimensional vector and input into the decoder along with the encoded features for decoding, resulting in a new one-dimensional vector. Then, the new one-dimensional vector can be rearranged to obtain the intermediate dynamic trajectory, which can be interpolated to obtain the target dynamic trajectory.

For example, if the first dynamic trajectory includes multiple first sub block trajectories, the multiple first sub block trajectories can be sorted in a predetermined order to obtain the first trajectory sequence, and then the encoded features and the first trajectory sequence can be decoded to obtain the target dynamic trajectory. Thus, by sorting multiple first sub block trajectories in a predetermined order and tiling the third dynamic trajectory into a one-dimensional first trajectory sequence before decoding, the processing requirements of the model can be met.

The predetermined order may be from left to right and from top to bottom, and the length of the first trajectory sequence is the same as the number of first sub block trajectories.

For example, when decoding the encoded features and the first trajectory sequence, the encoded features and the first trajectory sequence can be decoded to obtain a second trajectory sequence, and multiple second sub block trajectories in the second trajectory sequence can be rearranged to obtain a fourth dynamic trajectory. Then, the fourth dynamic trajectory can be interpolated to obtain the target dynamic trajectory.

The second trajectory sequence is one-dimensional data, and the length of the second trajectory sequence is the same as the number of second sub block trajectories.

Thus, by decoding the encoded features and the first trajectory sequence, a one-dimensional second trajectory sequence is obtained, which is then rearranged to obtain the fourth dynamic trajectory. Then, through interpolation processing, the target dynamic trajectory is obtained, which reduces the computational complexity of the model while improving the accuracy of dynamic trajectory prediction.

In step 206, an initial full atomic structure prediction model is trained based on a difference between the target dynamic trajectory and the first dynamic trajectory, to obtain the full atomic structure prediction model.

For example, based on the difference between the position information of an atom at any time point in the target dynamic trajectory and the position information of the same atom at any time point in the first dynamic trajectory, the first loss corresponding to any time point is determined. Based on the sum of the first losses corresponding to different time points, the second loss is obtained. Then, based on the second loss, the initial full atomic structure prediction model is trained to obtain the full atomic structure prediction model.

The first loss corresponding to any time point can be the sum of the sub losses corresponding to respective atoms, and the sub losses corresponding to each atom can be determined based on the difference between the position information of each atom in the target dynamic trajectory and the position information of the same atom in the first dynamic trajectory.

Therefore, by training the initial full atomic structure prediction model based on the difference between the position information of atoms at each time point in the target dynamic trajectory and the position information of the same atoms at each time point in the first dynamic trajectory, the full atomic structure prediction model is obtained, which improves the accuracy of the model.

In embodiments of the present disclosure, the third dynamic trajectory is obtained by performing dimensionality reduction on the second dynamic trajectory, and the encoded features and the third dynamic trajectory are decoded to obtain the target dynamic trajectory. Therefore, reducing the dimensionality of the second dynamic trajectory before using it for decoding can reduce computational complexity and improve model processing speed.

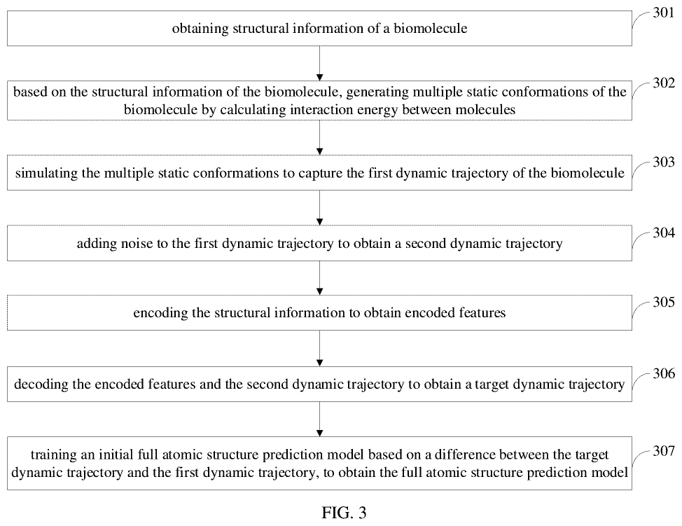

FIG. 3 is a schematic flowchart of a training method for a full atomic structure prediction model provide in another embodiment of the present disclosure.

As shown in FIG. 3, the training method for the full atomic structure prediction model includes followings.

In step 301, structural information of a biomolecule is obtained.

In this disclosure, step 301 can be implemented using any manner in embodiments of the present disclosure, and will not be elaborated here.

In step 302, based on the structural information of the biomolecule, multiple static conformations of the biomolecule are generated by calculating interaction energy between molecules.

In this disclosure, physics based docking software is used to generate multiple static conformations of the biomolecule by calculating the interaction energy between molecules based on the structural information of the biomolecule. These static conformations cover different binding modes and orientations, greatly enriching the diversity of data.

For example, the biomolecule is a complex of proteins and small molecules. Docking software can be used to predict and generate multiple static conformations of the complex of proteins and small molecules by calculating the interaction energy between molecules based on the sequence information of proteins and small molecule information.

In step 303, the multiple static conformations are simulated to capture the first dynamic trajectory of the biomolecule.

In this disclosure, molecular dynamics simulation software can be applied to simulate multiple static conformations for a long time and capture the first dynamic trajectory of the biomolecule. The first dynamic trajectory not only demonstrates the flexibility of the biomolecule, but also provides rich dynamic interaction information, which helps the model learn more realistic biomolecule behavior.

For example, it is possible to simulate multiple static conformations of a complex of multiple biomolecules for a long time, capturing the dynamic behavior of the complex of biomolecules in a solvent environment, such as conformational changes, dynamic trajectories, etc.

In step 304, noise is added to the first dynamic trajectory, to obtain a second dynamic trajectory.

In this disclosure, step 304 can be implemented using any manner in embodiments of the present disclosure, and will not be elaborated here.

In step 305, the structural information is encoded to obtain encoded features.

In this disclosure, step 305 can be implemented using any manner in embodiments of the present disclosure, and will not be elaborated here.

In step 306, the encoded features and the second dynamic trajectory are decoded to obtain a target dynamic trajectory.

In this disclosure, step 306 can be implemented using any manner in embodiments of the present disclosure, and will not be elaborated here.

For example, the position information of atoms in the second dynamic trajectory is sorted in a predetermined order to obtain the third trajectory sequence, and the encoded features and the third trajectory sequence are decoded to obtain the fourth trajectory sequence. Then, the position information of the atoms in the fourth trajectory sequence is rearranged to obtain the target dynamic trajectory. Therefore, converting the second dynamic trajectory into a one-dimensional third trajectory sequence before decoding can meet the processing requirements of the model.

In step 307, an initial full atomic structure prediction model is trained based on a difference between the target dynamic trajectory and the first dynamic trajectory, to obtain the full atomic structure prediction model.

In this disclosure, step 307 can be implemented using any manner in embodiments of the present disclosure, and will not be elaborated here.

In embodiments of the present disclosure, multiple static conformations of the biomolecule are generated by calculating the interaction energy between molecules. Due to the fact that static conformations cover different binding modes and orientations, the diversity of data is greatly enriched. Therefore, the first dynamic trajectory obtained based on multiple static conformations not only demonstrates the flexibility of biomolecule, but also provides rich dynamic interaction information, which helps the model learn more realistic biomolecule behavior. Based on the first dynamic trajectory, the full atomic structure prediction model is trained, which can capture temporal dependence and complex interactions from the first dynamic trajectory, thereby improving the accuracy of structure prediction and achieving high-precision full atomic structure prediction.

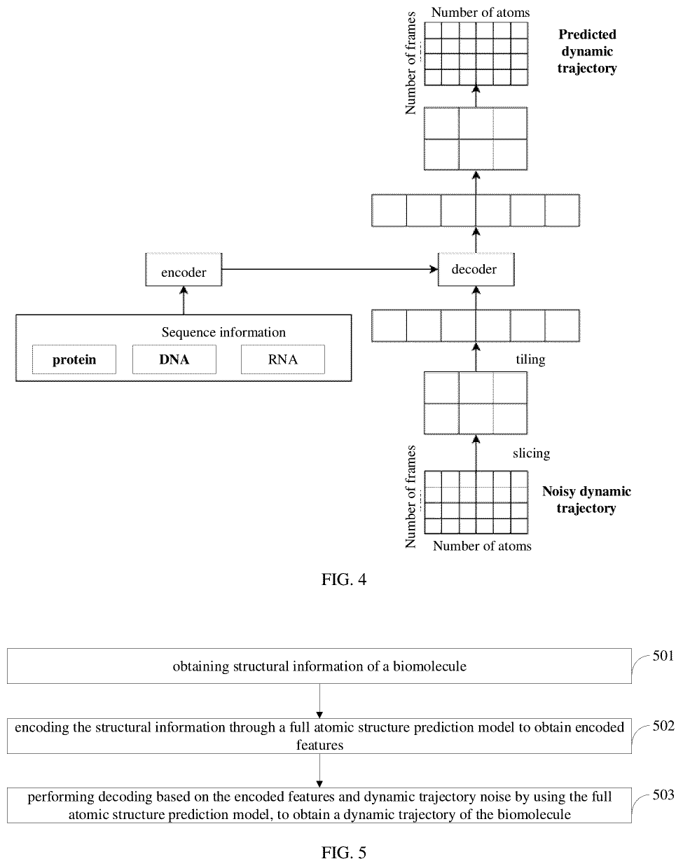

For ease of understanding, the following explanation will be made in conjunction with FIG. 4, which is a schematic diagram of the process of dynamic trajectory prediction using a full atomic structure prediction model provided in an embodiment of the present disclosure.

As shown in FIG. 4, the full atomic structure prediction model includes an encoder and a decoder. Taking the complex of biomolecules such as proteins, DNA, and RNA as an example, the encoder can be used to encode the sequence information of proteins, DNA, RNA, etc., obtain encoded features, and the encoded features are input into the decoder.

In addition, noise is added to the dynamic trajectory samples of the complex to obtain the noisy dynamic trajectory. By segmenting the noisy dynamic trajectory of the complex, the dimension of the noisy dynamic trajectory is reduced, and then flattened into a one-dimensional vector, which is input to the decoder. The decoder decodes the encoded features and one-dimensional vector to obtain a new one-dimensional vector, rearranges the new one-dimensional vector, and then obtains the predicted dynamic trajectory through interpolation.

The shape of the noisy dynamic trajectory can be represented as number of frames*number of atoms*3, where 3 represents three dimensions in the three-dimensional coordinate information. In FIG. 4, each block of the noisy dynamic trajectory can represent the position information of an atom, and the sequence information corresponds to the dynamic trajectory. For example, the sequence information of proteins refers to the arrangement order of amino acids in the protein molecule, and the atoms in each frame of the dynamic trajectory are arranged according to the arrangement order of amino acids. If it is a complex, the atoms of one biomolecule can be sorted and then the atoms of another biomolecule can be sorted, without any restriction on the order of biomolecules.

During the model training phase, the initial full atomic structure prediction model can be trained based on the difference between the predicted dynamic trajectory and the dynamic trajectory samples to obtain the full atomic structure prediction model.

In order to implement the above embodiments, embodiments of the present disclosure also propose a method for predicting a full atomic structure. FIG. 5 is a flowchart of a method for predicting a full atomic structure provided in an embodiment of the present disclosure.

As shown in FIG. 5, the method for predicting the full atomic structure includes followings.

In step 501, structural information of a biomolecule is obtained.

In this disclosure, the explanation of the structural information of the biomolecule can be found in the above embodiments, which will not be elaborated here.

In step 502, the structural information is encoded using a full atomic structure prediction model to obtain encoded features.

The full atomic structure prediction model can be trained using the training method described in the above embodiments.

The full atomic structure prediction model can include an encoder and a decoder. The structural information can be input into the encoder to encode the structural information of the biomolecule, to obtain encoded features.

In step 503, through the full atomic structure prediction model, decoding is performed according to the encoded features and dynamic trajectory noise to obtain the dynamic trajectory of the biomolecule.

The dynamic trajectory noise can include noise at different time points, such as noise from multiple frames.

In this disclosure, the encoded features and dynamic trajectory noise can be input into the decoder for decoding, and the noise can be gradually removed through decoding to obtain the dynamic trajectory of the biomolecule. The dynamic trajectory can include the position information of atoms in the biomolecule at different time points.

In embodiments of the present disclosure, the full atomic structure prediction model trained using the training method described in the above embodiments can predict the dynamic trajectory of the biomolecule and improve the accuracy of prediction.

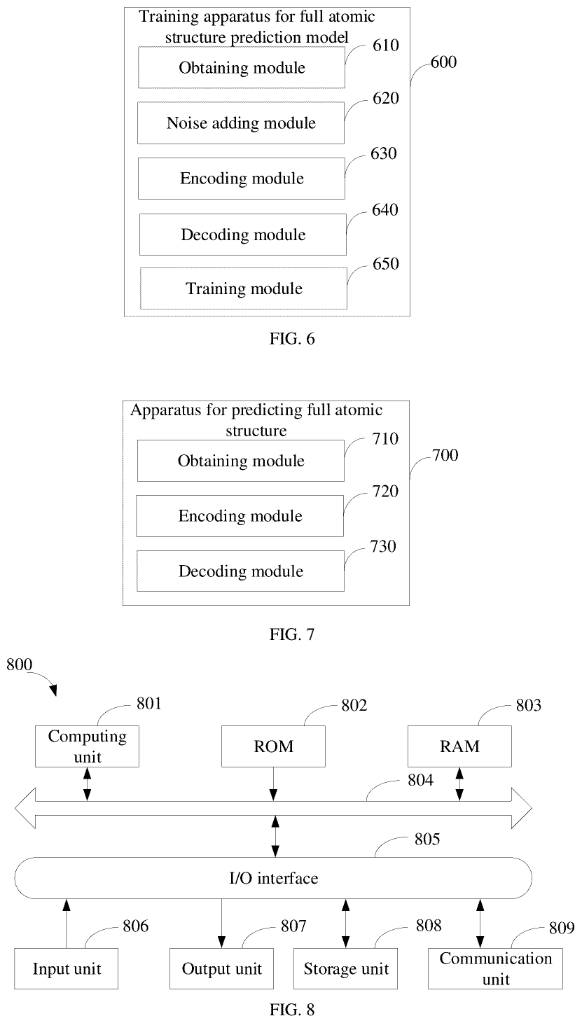

In order to implement the above embodiments, the present disclosure also proposes a training apparatus for a full atomic structure prediction model. FIG. 6 is a block diagram of a training apparatus for a full atomic structure prediction model provided in an embodiment of the present disclosure.

As shown in FIG. 6, the training apparatus 600 for the full atomic structure prediction model includes:

an obtaining module 610, configured to obtain structural information of a biomolecule and a first dynamic trajectory of the biomolecules; wherein, the first dynamic trajectory includes position information of atoms in the biomolecule at different time points; a noise adding module 620, configured to add noise to the first dynamic trajectory to obtain a second dynamic trajectory;

an encoding module 630, configured to encode the structural information to obtain encoded features;

a decoding module 640, configured to decode the encoded features and the second dynamic trajectory to obtain a target dynamic trajectory; and

a training module 650, configured to train an initial full atomic structure prediction model based on a difference between the target dynamic trajectory and the first dynamic trajectory, to obtain the full atomic structure prediction model.

Optionally, the decoding module 640 is configured to:
perform dimensionality reduction on the second dynamic trajectory to obtain a third dynamic trajectory; and
decode the encoded features and the third dynamic trajectory to obtain the target dynamic trajectory.

Optionally, the decoding module 640 is configured to:
sort the multiple first sub block trajectories in a predetermined order to obtain a first trajectory sequence; and
decode the encoded features and the first trajectory sequence to obtain the target dynamic trajectory.

Optionally, the decoding module 640 is configured to:
decode the encoded features and the first trajectory sequence to obtain a second trajectory sequence;
rearrange multiple second sub block trajectories in the second trajectory sequence to obtain a fourth dynamic trajectory; and
interpolate the fourth dynamic trajectory to obtain the target dynamic trajectory.

Optionally, the obtaining module 610 is configured to:
based on the structural information of the biomolecule, generate multiple static conformations of the biomolecule by calculating interaction energy between molecules; and
simulate the multiple static conformations to capture the first dynamic trajectory of the biomolecule.

Optionally, the decoding module 640 is configured to:
sort the position information of atoms in the second dynamic trajectory in a predetermined order, to obtain a third trajectory sequence;
decode the encoded features and the third trajectory sequence to obtain a fourth trajectory sequence; and
rearrange the position information of atoms in the fourth trajectory sequence to obtain the target dynamic trajectory.

Optionally, the training module 650 is configured to:
determine a first loss corresponding to any time point based on a difference between the position information of an atom at any time point in the target dynamic trajectory and the position information of the same atom at any time point in the first dynamic trajectory;
obtain a second loss based on the first losses corresponding to different time points; and
train the initial full atomic structure prediction model based on the second loss, to obtain the full atomic structure prediction model.

It should be noted that the explanation of the embodiments of the training method for the full atomic structure prediction model mentioned above also applies to the training apparatus for the full atomic structure prediction model in this embodiment, so it will not be repeated here.

In embodiments of the present disclosure, encoded features are obtained by encoding the structural information of the biomolecule, and the second dynamic trajectory obtained by adding noise to the first dynamic trajectory and the encoded features are decoded to obtain the target dynamic trajectory. Then, based on the difference between the target dynamic trajectory and the first dynamic trajectory, the full atomic structure prediction model is trained. Therefore, based on the structural information of the biomolecule and the first dynamic trajectory of the biomolecule, the full atomic structure prediction model that can predict the dynamic trajectory of the biomolecule is obtained by training, achieving prediction of the dynamic trajectory of the biomolecule and improving the generalization ability of the model.

In order to achieve the above embodiments, embodiments of the present disclosure further provide an apparatus for predicting a full atomic structure. FIG. 7 is a block diagram of an apparatus for predicting a full atomic structure provided in an embodiment of the present disclosure.

As shown in FIG. 7, the apparatus 700 for predicting a full atomic structure includes: an obtaining module 710, configured to obtain structural information of a biomolecule; an encoding module 720, configured to encode the structural information through a full atomic structure prediction model to obtain encoded features; wherein, the full atomic structure prediction model can be trained using the method described in the above embodiments;

a decoding module 730, configured to perform decoding based on the encoded features and dynamic trajectory noise by using the full atomic structure prediction model, to obtain a dynamic trajectory of the biomolecule; wherein, the dynamic trajectory comprises position information of atoms in the biomolecule at different time points.

It should be noted that the explanation of the embodiments of the method for predicting the full atomic structure mentioned above also applies to the apparatus for predicting the full atomic structure in this embodiment, so it will not be repeated here.

In embodiments of the present disclosure, the full atomic structure prediction model trained using the training method described in the above embodiments can predict the dynamic trajectory of the biomolecule and improve the accuracy of prediction.

According to embodiments of the disclosure, the disclosure also provides an electronic device, a readable storage medium and a computer program product.

FIG. 8 is a block diagram of an electronic device 800 used to implement embodiments of the disclosure. Electronic devices are intended to represent various forms of digital computers, such as laptop computers, desktop computers, workbenches, personal digital assistants, servers, blade servers, mainframe computers, and other suitable computers. Electronic devices may also represent various forms of mobile devices, such as personal digital processing, cellular phones, smart phones, wearable devices, and other similar computing devices. The components shown here, their connections and relations, and their functions are merely examples, and are not intended to limit the implementation of the disclosure described and/or required herein.

As illustrated in FIG. 8, the electronic device 800 includes a computing unit 801 that can perform various appropriate actions and processes based on computer programs stored in ROM (Read Only Memory) 802 or computer programs loaded from a storage unit 808 into RAM (Random Access Memory) 803. In RAM 803, various programs and data required for the operation of the device 800 can also be stored. The computing unit 801, ROM 802, and RAM 803 are connected to each other through a bus 804. The I/O (Input/Output) interface 805 is also connected to the bus 804.

Multiple components in device 800 are connected to the I/O interface 805, including input unit 806 such as keyboard, mouse, etc; output unit 807, such as various types of displays, speakers, etc; storage unit 808, such as disks, CDs, etc; and communication unit 809, such as network card, modem, wireless communication transceiver, etc. The communication unit 809 allows device 800 to exchange information/data with other devices through computer networks such as the Internet and/or various telecommunications networks.

The computing unit 801 can be various general-purpose and/or specialized processing components with processing and computing capabilities. Some examples of computing unit 801 include but are not limited to CPU (Central Processing Unit), GPU (Graphic Processing Units), various specialized AI (Artificial Intelligence) computing chips, various computing units that run machine learning model algorithms, DSP (Digital Signal Processor), and any suitable processor, controller, microcontroller, etc. The computing unit 801 executes various methods and processes described above, such as the training method for the full atomic structure prediction model. For example, in some embodiments, the training method for the full atomic structure prediction model may be implemented as a computer software program tangibly contained in a machine-readable medium, such as storage unit 808. In some embodiments, some or all of the computer program may be loaded and/or installed onto the device 800 via ROM 802 and/or communication unit 809. When the computer program is loaded into RAM 803 and executed by the computing unit 801, one or more steps of the training method for the full atomic structure prediction model described above can be performed. Alternatively, in other embodiments, the computing unit 801 may be configured to perform the training method for the full atomic structure prediction model through any other suitable means (e.g., with the aid of firmware).

Various embodiments of the systems and technologies described herein may be implemented in digital electronic circuit systems, integrated circuit systems, Application Specific Integrated Circuits (ASICs), computer hardware, firmware, software, and/or combinations thereof. These various embodiments may be implemented in one or more computer programs, which may be executed and/or interpreted on a programmable system including at least one programmable processor. The programmable processor may be dedicated or general purpose programmable processor that receives data and instructions from a storage system, at least one input device, and at least one output device, and transmits the data and instructions to the storage system, the at least one input device, and the at least one output device.

The program codes used to implement the method of the present disclosure can be written in any combination of one or more programming languages. These program codes can be provided to processors or controllers of general-purpose computers, specialized computers, or other programmable data processing devices, so that when executed by the processor or controller, the program codes implement the functions/operations specified in the flowchart and/or block diagram. The program codes can be executed entirely on a machine, partially on a machine, partially on a machine as a standalone software package and partially on a remote machine, or entirely on a remote machine or server.

In the context of this disclosure, a machine readable medium may be a tangible medium that contains or stores programs for use by or in combination with an instruction execution system, apparatus, or device. The machine readable medium can be machine readable signal medium or machine readable storage medium. The machine readable medium may include, but are not limited to, electronic, magnetic, optical, electromagnetic, infrared, or semiconductor systems, devices, or equipment, or any suitable combination of the above. More specific examples of machine-readable storage medium include electrical connections based on one or more wires, portable computer disks, hard drives, RAM, ROM, EPROM (Electrically Programmable Read Only Memory) or flash memory, fiber optics, CD-ROM (Compact Disc Read Only Memory), optical storage devices, magnetic storage devices, or any suitable combination of the above.

In order to provide interaction with a user, the systems and techniques described herein may be implemented on a computer having a display device (e.g., a Cathode Ray Tube (CRT) or a LCD monitor for displaying information to a user); and a keyboard and pointing device (such as a mouse or trackball) through which the user can provide input to the computer. Other kinds of devices may also be used to provide interaction with the user. For example, the feedback provided to the user may be any form of sensory feedback (e.g., visual feedback, auditory feedback, or haptic feedback), and the input from the user may be received in any form (including acoustic input, voice input, or tactile input).

The systems and technologies described herein can be implemented in a computing system that includes background components (for example, a data server), or a computing system that includes middleware components (for example, an application server), or a computing system that includes front-end components (for example, a user computer with a graphical user interface or a web browser, through which the user can interact with the implementation of the systems and technologies described herein), or a computing system that includes any combination of such background components, intermediate computing components, or front-end components. The components of the system may be interconnected by any form or medium of digital data communication (e.g., a communication network). Examples of communication networks include: a local area network (LAN), a wide area network (WAN), and the Internet.

The computer system may include a client and a server. The client and server are generally remote from each other and interacting through a communication network. The client-server relation is generated by computer programs running on the respective computers and having a client-server relation with each other. The server may be a cloud server, also known as a cloud computing server or a cloud host, which is a host product in the cloud computing service system, to solve the defects of difficult management and weak business scalability in the traditional physical host and Virtual Private Server (VPS) service. The server can also be a server of distributed system or a server combined with block-chain.

It should be noted that the above electronic device can implement the method for predicting the full atomic structure described in the above embodiments.

According to embodiments of the present disclosure, the disclosure further provides a computer program product. When the instructions in the computer program product are executed by the processor, the training method for the full atomic structure prediction model or the method for predicting the full atomic structure proposed in the above embodiments of the present disclosure is implemented.

It should be understood that the various forms of processes shown above can be used to reorder, add or delete steps. For example, the steps described in the disclosure could be performed in parallel, sequentially, or in a different order, as long as the desired result of the technical solution disclosed in the disclosure is achieved, which is not limited herein. The above specific embodiments do not constitute a limitation on the protection scope of the disclosure. Those skilled in the art should understand that various modifications, combinations, sub-combinations and substitutions can be made according to design requirements and other factors. Any modification, equivalent replacement and improvement made within the spirit and principle of the disclosure shall be included in the protection scope of the disclosure.

1. A training method for a full atomic structure prediction model, comprising:
obtaining structural information of a biomolecule and a first dynamic trajectory of the biomolecule; wherein, the first dynamic trajectory comprises position information of atoms in the biomolecule at different time points;
adding noise to the first dynamic trajectory to obtain a second dynamic trajectory;
encoding the structural information to obtain encoded features;
decoding the encoded features and the second dynamic trajectory to obtain a target dynamic trajectory; and
training an initial full atomic structure prediction model based on a difference between the target dynamic trajectory and the first dynamic trajectory, to obtain the full atomic structure prediction model.

2. The method of claim 1, wherein decoding the encoded features and the second dynamic trajectory to obtain the target dynamic trajectory comprises:
performing dimensionality reduction on the second dynamic trajectory to obtain a third dynamic trajectory; and
decoding the encoded features and the third dynamic trajectory to obtain the target dynamic trajectory.

3. The method of claim 2, wherein the third dynamic trajectory comprises multiple first sub block trajectories, and decoding the encoded features and the third dynamic trajectory to obtain the target dynamic trajectory comprises:
sorting the multiple first sub block trajectories in a predetermined order to obtain a first trajectory sequence; and
decoding the encoded features and the first trajectory sequence to obtain the target dynamic trajectory.

4. The method of claim 3, wherein decoding the encoded features and the first trajectory sequence to obtain the target dynamic trajectory comprises:
decoding the encoded features and the first trajectory sequence to obtain a second trajectory sequence;
rearranging multiple second sub block trajectories in the second trajectory sequence to obtain a fourth dynamic trajectory;
interpolating the fourth dynamic trajectory to obtain the target dynamic trajectory.

5. The method of claim 1, wherein obtaining the first dynamic trajectory of the biomolecule, comprises:
based on the structural information of the biomolecule, generating multiple static conformations of the biomolecule by calculating interaction energy between molecules; and
simulating the multiple static conformations to capture the first dynamic trajectory of the biomolecule.

6. The method of claim 1, wherein decoding the encoded features and the second dynamic trajectory to obtain the target dynamic trajectory comprises:
sorting the position information of atoms in the second dynamic trajectory in a predetermined order, to obtain a third trajectory sequence;
decoding the encoded features and the third trajectory sequence to obtain a fourth trajectory sequence; and
rearranging the position information of atoms in the fourth trajectory sequence to obtain the target dynamic trajectory.

7. The method of claim 1, wherein training the initial full atomic structure prediction model based on the difference between the target dynamic trajectory and the first dynamic trajectory, to obtain the full atomic structure prediction model, comprises:
determining a first loss corresponding to any time point based on a difference between the position information of an atom at any time point in the target dynamic trajectory and the position information of the same atom at any time point in the first dynamic trajectory;
obtaining a second loss based on the first losses corresponding to different time points; and
training the initial full atomic structure prediction model based on the second loss, to obtain the full atomic structure prediction model.

8. A method for predicting a full atomic structure, comprising:
obtaining structural information of a biomolecule;
encoding the structural information through a full atomic structure prediction model to obtain encoded features; wherein, the full atomic structure prediction model is trained by:
obtaining structural information of a biomolecule and a first dynamic trajectory of the biomolecule; wherein, the first dynamic trajectory comprises position information of atoms in the biomolecule at different time points;
adding noise to the first dynamic trajectory to obtain a second dynamic trajectory;
encoding the structural information to obtain encoded features;
decoding the encoded features and the second dynamic trajectory to obtain a target dynamic trajectory; and
training an initial full atomic structure prediction model based on a difference between the target dynamic trajectory and the first dynamic trajectory, to obtain the full atomic structure prediction model; and
performing decoding based on the encoded features and dynamic trajectory noise by using the full atomic structure prediction model, to obtain a dynamic trajectory of the biomolecule; wherein, the dynamic trajectory comprises position information of atoms in the biomolecule at different time points.

9. The method of claim 8, wherein decoding the encoded features and the second dynamic trajectory to obtain the target dynamic trajectory comprises:
performing dimensionality reduction on the second dynamic trajectory to obtain a third dynamic trajectory; and
decoding the encoded features and the third dynamic trajectory to obtain the target dynamic trajectory.

10. The method of claim 9, wherein the third dynamic trajectory comprises multiple first sub block trajectories, and decoding the encoded features and the third dynamic trajectory to obtain the target dynamic trajectory comprises:
sorting the multiple first sub block trajectories in a predetermined order to obtain a first trajectory sequence; and
decoding the encoded features and the first trajectory sequence to obtain the target dynamic trajectory.

11. The method of claim 10, wherein decoding the encoded features and the first trajectory sequence to obtain the target dynamic trajectory comprises:
decoding the encoded features and the first trajectory sequence to obtain a second trajectory sequence;
rearranging multiple second sub block trajectories in the second trajectory sequence to obtain a fourth dynamic trajectory;
interpolating the fourth dynamic trajectory to obtain the target dynamic trajectory.

12. The method of claim 8, wherein obtaining the first dynamic trajectory of the biomolecule, comprises:
based on the structural information of the biomolecule, generating multiple static conformations of the biomolecule by calculating interaction energy between molecules; and
simulating the multiple static conformations to capture the first dynamic trajectory of the biomolecule.

13. The method of claim 8, wherein decoding the encoded features and the second dynamic trajectory to obtain the target dynamic trajectory comprises:
sorting the position information of atoms in the second dynamic trajectory in a predetermined order, to obtain a third trajectory sequence;
decoding the encoded features and the third trajectory sequence to obtain a fourth trajectory sequence; and
rearranging the position information of atoms in the fourth trajectory sequence to obtain the target dynamic trajectory.

14. The method of claim 8, wherein training the initial full atomic structure prediction model based on the difference between the target dynamic trajectory and the first dynamic trajectory, to obtain the full atomic structure prediction model, comprises:
determining a first loss corresponding to any time point based on a difference between the position information of an atom at any time point in the target dynamic trajectory and the position information of the same atom at any time point in the first dynamic trajectory;
obtaining a second loss based on the first losses corresponding to different time points; and
training the initial full atomic structure prediction model based on the second loss, to obtain the full atomic structure prediction model.

15. An electronic device, comprising:
at least one processor; and
a memory communicatively coupled to the at least one processor; wherein,
the memory stores instructions executable by the at least one processor, when the instructions are executed by the at least one processor, the at least one processor is configured to:
obtain structural information of a biomolecule and a first dynamic trajectory of the biomolecule; wherein, the first dynamic trajectory comprises position information of atoms in the biomolecule at different time points;
add noise to the first dynamic trajectory to obtain a second dynamic trajectory;
encode the structural information to obtain encoded features;
decode the encoded features and the second dynamic trajectory to obtain a target dynamic trajectory; and
train an initial full atomic structure prediction model based on a difference between the target dynamic trajectory and the first dynamic trajectory, to obtain the full atomic structure prediction model.

16. The electronic device of claim 15, wherein the at least one processor is configured to:
perform dimensionality reduction on the second dynamic trajectory to obtain a third dynamic trajectory; and
decode the encoded features and the third dynamic trajectory to obtain the target dynamic trajectory.

17. The electronic device of claim 16, wherein the at least one processor is configured to:
sort the multiple first sub block trajectories in a predetermined order to obtain a first trajectory sequence; and
decode the encoded features and the first trajectory sequence to obtain the target dynamic trajectory.

18. An electronic device, comprising:
at least one processor; and
a memory communicatively coupled to the at least one processor; wherein,
the memory stores instructions executable by the at least one processor, when the instructions are executed by the at least one processor, the at least one processor is configured to perform the method of claim 8.

19. A non-transitory computer-readable storage medium having computer instructions stored thereon, wherein the computer instructions are configured to enable a computer to implement the method according to claim 1.

20. A non-transitory computer-readable storage medium having computer instructions stored thereon, wherein the computer instructions are configured to enable a computer to implement the method according to claim 8.
